(19) United States
(12) Patent Application Publication
(10) Pub. No.: US 2021/0308290 A1
(43) Pub. Date: Oct. 7, 2021

(54) HAND SANITIZER DISPENSER

(71) Applicant: Origyn LLC, Boston, MA (US)

(72) Inventor: Jason Thorne, Dover, MA (US)

(73) Assignee: Origyn LLC, Boston, MA (US)

(21) Appl. No.: 17/221,506

(22) Filed: Apr. 2, 2021

Related U.S. Application Data
(60) Provisional application No. 63/005,128, filed on Apr. 3, 2020.
(60) Provisional application No. 63/069,394, filed on Aug. 24, 2020.

(51) Int. Cl. A61L 2/00; A61L 2/24

(52) U.S. Cl. CPC A61L 2/0088; A61L 2202/14; A61L 2202/15; A61L 2/24

(57) ABSTRACT

A system and method of use are provided for a hand sanitizer dispenser bottle. Disclosed herein is a system comprising a hand sanitizer dispenser bottle, including a reservoir configured to contain sanitizing liquid, an arm extending from the reservoir, a nozzle positioned on a lower surface of an upper portion of the arm, the nozzle configured for dispensing sanitizing liquid, and a sensor for detection the presence of a user's hand. In some embodiments, the nozzle is a dual-headed nozzle.

### CROSS-REFERENCE TO RELATED APPLICATIONS

This application claim benefit of priority under 35 U.S.C. § 119(e) to U.S. Provisional Application Ser. No. 63/069,394, titled “HAND SANITIZER DISPENSER” and filed on Aug. 24, 2020 and U.S. Provisional Application Ser. No. 63/005,128, titled “HAND SANITIZER DISPENSER” and filed on Apr. 3, 2020, each of which are hereby incorporated by reference in their entireties for all purposes.

### TECHNICAL FIELD

The present disclosure relates generally to a dispenser and more specifically to a hand sanitizer dispenser.

### BACKGROUND

Germs and viruses can be transmitted easily from person to person from germs carried from one's hands and placed on a surface of a common area, such as a handrail, door handle, knob, or countertop.

### SUMMARY

In a first example, system is provided comprising a hand sanitizer dispenser, including a reservoir configured to contain sanitizing liquid, an arm extending above the reservoir, a nozzle positioned on a lower surface of an upper portion of the arm, the nozzle configured for dispensing sanitizing liquid, and a sensor for detection the presence of a user's hand.

Example 2 includes the subject matter of Example 1, wherein the nozzle is a dual headed nozzle.

Example 3 includes the subject matter of Example 2, wherein the dual headed nozzle comprises a pair of fan shaped protrusion configured to dispense sanitizing liquid.

Example 4 includes the subject matter of Example 1, further comprising a three-position button positioned on the arm.

Example 5 includes the subject matter of Example 4, wherein the three-position button is configured to switch between a set of dispensing modes, wherein each dispensing mode is targeted at one of liquid, gel, or foam.

Example 6 includes the subject matter of Example 1, wherein the nozzle is positioned inside a spray head.

Example 7 includes the subject matter of Example 3, further comprising a cap on an upper portion of the reservoir.

Example 8 includes the subject matter of Example 7, wherein a ball valve positioned in a seal is configured to facilitate a fluid-tight connection of the cap into a housing of the hand sanitizer dispenser.

Example 9 includes the subject matter of Example 8, wherein the ball valve is configured to minimize backflow of the sanitizing liquid.

Example 10 includes the subject matter of Example 7, further comprising tubing extending from the cap.

Example 11 includes the subject matter of Example 10, further comprising a one-way valve at the end of the tubing.

Example 12 includes the subject matter of Example 11, wherein the one-way valve comprises at least one of a mesh screen, a seal, and a spring.

Example 13 includes the subject matter of Example 8, further comprising a support post and a stand.

Example 14 includes the subject matter of Example 13, wherein at least one of the housing, the reservoir, the support post, and the stand are configured to be connected via a screw connection.

In Example 15, a method for dispensing sanitizing liquid is provided, comprising providing a hand sanitizer dispenser according to Example 1, sensing the presence of a user's hand; and dispensing sanitizing liquid through the nozzle.

Example 16 includes the subject matter of Example 15, wherein the nozzle is a dual-headed nozzle.

Example 17 includes the subject matter of Example 15, further comprising preventing a pump within the hand sanitizer dispenser from losing a strength of dispersal.

Example 18 includes the subject matter of Example 15, further comprising minimizing dripping during removal of a reservoir from the hand sanitizer dispenser.

In Example 19 a method for dispensing sanitizing liquid is provided, comprising providing a hand sanitizer dispenser comprising a nozzle, receiving user input from a button, and dispensing sanitizing liquid through the nozzle upon receipt of user input.

The features and advantages described herein are not all-inclusive and, in particular, many additional features and advantages will be apparent to one of ordinary skill in the art in view of the drawings, specification, and claims. Moreover, it should be noted that the language used in the specification has been principally selected for readability and instructional purposes, and not to limit the scope of the subject matter described herein.

### BRIEF DESCRIPTION OF THE FIGURES

The drawings described herein are for illustration purposes only and are not intended to limit the scope of the present disclosure in any way.

FIG. 1 illustrates a perspective view of a hand sanitizer dispenser, in accordance with one embodiment of the disclosure.

FIG. 2 illustrates a side view of a hand sanitizer dispenser, in accordance with one embodiment of the disclosure.

FIG. 3 illustrates a perspective view of a hand sanitizer dispenser including a button positioned on the arm configured for activating dispensing of sanitizing liquid, in accordance with one embodiment of the disclosure.

FIG. 4 illustrates a perspective view of a nozzle of a hand sanitizer dispenser, in accordance with one embodiment of the disclosure.

FIG. 5 illustrates a top view of a hand sanitizer dispenser, in accordance with one embodiment of the disclosure.

FIG. 6 illustrates a reservoir for containing hand sanitizer, in accordance with one embodiment of the disclosure.

FIG. 7 illustrates a perspective interior view of a hand sanitizer dispenser, in accordance with one embodiment of the disclosure.

FIG. 8 illustrates a cross-sectional view of a one-way valve, in accordance with one embodiment of the disclosure.

FIGS. 9A-C illustrate side views of a hand sanitizer dispenser, in accordance with one embodiment of the disclosure.

FIG. 10A illustrates a front interior view of a hand sanitizer dispenser, in accordance with one embodiment of the disclosure.

FIG. 10B illustrates a magnified view of a connection point between a reservoir and hand sanitizer dispenser housing from FIG. 10A, in accordance with one embodiment of the disclosure.

These and other features of the present embodiments will be understood better by reading the following detailed description, taken together with the figures herein described. The accompanying drawings are not intended to be drawn to scale. For purposes of clarity, not every component may be labeled in every drawing.

### DETAILED DESCRIPTION

Embodiments are presented herein for a hand sanitizer dispenser. Properly disinfecting one's hands is essential to the prevention of disease transmission. Features are disclosed herein to encourage proper disinfection of a user's hands after a single use of the hand sanitizer dispenser.

In some embodiments, a sensor is positioned at a bend of an arm extending between the bottle containing sanitizing liquid and the nozzle. The sensor is configured to detect if a user is present in front of the nozzle and configured to dispense sanitizing liquid when a user is detected.

The hand sanitizer dispenser is designed to be cordless and operate on battery power. The hand sanitizer dispenser is designed to be portable and easily transported between school, work, home, and/or any public area.

The hand sanitizer reservoir is configured to be compatible with various sanitizing liquids. For example, liquid, gel, or foam can be put into the reservoir for use with the system.

In some embodiments, a button is provided that is configured to switch between different dispensing times. For example, a three-position button that is configured to switch between three positions to adjust the dispense time depending on the type of dispensing liquid provided in the reservoir. In one embodiment, a first dispensing time would apply to a liquid sanitizing liquid, a second dispensing time would apply to a gel sanitizing liquid, and a third dispensing time would apply to a foam sanitizing liquid. In some embodiments, the hand sanitizing dispenser is configured dispense liquid spray. In some embodiments, the hand sanitizer dispenser is configured to disperse a liquid spray over a wide area. Liquid spray quickly provides wide coverage of a user's hands for disinfection. In some embodiments, the apparatus described herein is configured to dispense 75% liquid ethyl alcohol.

In some embodiments, the arm is rigid and arcuate. In some embodiments, the arm is a goose neck arm configured to provide optimized clearance for hands and fingers as they approach the nozzle for dispensing sanitizing liquid. Improved clearance near the nozzle means you can spray a larger area of one or both of a user's hands with more spray output in less time. Various features of the disclosure provide an optimization of surface area coverage with the sanitizing spray for disinfection. For example, the nozzle is configured to provide wide coverage and maximize the amount of sanitizing liquid dispensed. For example, the position of the nozzle above the reservoir, as opposed to in front of the reservoir, provides improved clearance for one or both of a user's hands.

In some embodiments, a dual headed spray nozzle is provided. A dual headed spray nozzle is configured to provide coverage to a pair of hands simultaneously. A dual headed spray nozzle is configured to maximize sanitizing liquid coverage on the skin surface of a user. For example, a dual headed spray nozzle comprises two fan shaped protrusions extending in opposite directions from a lower surface of the upper portion of the arm of the hand sanitizer dispenser. Accordingly, the apparatus and method disclosed herein are configured to maximize disinfection and minimize any remaining bacteria or viruses left behind following use.

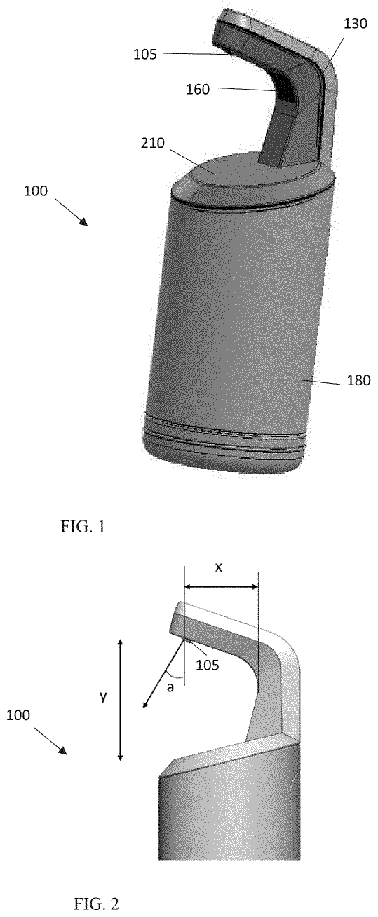

The components and operation of some embodiments of the hand sanitizer dispenser are presented in FIGS. 1-10B.

FIG. 1 details a perspective view of hand sanitizer dispenser 100, in accordance with an embodiment of the disclosure. Arm 130 extends from reservoir 180, which contains sanitizing liquid 170. Arm 130 is configured with sensor 160 for detecting the presence of a user's hand. Sensor 160 is a motion detector, in the illustrated embodiment. In some embodiments, sensor 160 is an infrared sensor. An angled surface 210 is shown at the top portion of reservoir 180. Angled surface 210 is configured to maximize the space for a user's fingers or hands to receive sanitizing liquid. Nozzle 105 is positioned on a lower surface of the upper portion of arm 130 of hand sanitizer dispenser 100. In the illustrated embodiment, the position of the arm 130 is above angled surface 210. Thus, sanitizing liquid 170 is dispensed above reservoir 180.

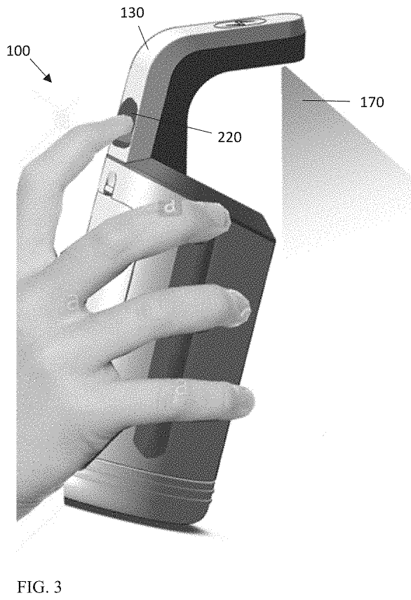

FIG. 2 illustrates a side view of hand sanitizer dispenser 100, in accordance with one embodiment of the disclosure. In some embodiments, the x dimension is greater than 1″, between 1″ and 3″, between 1.5″ and 2.5″, or smaller than 3″. In some embodiments, the y dimension is greater than 1.5″, between 1.5″ and 3.5″, between 2″ and 3″, or less than 3.5″. In some embodiments, angle a is between 0° and 45°, between 15° and 40°, or between 25° and 35°. The illustrated embodiment of FIG. 2 highlights the forward angle a of nozzle 105. Nozzle 105 is configured to spray optimally into the center of a user's hands to maximize the disinfection provided by sanitizing liquid 170.

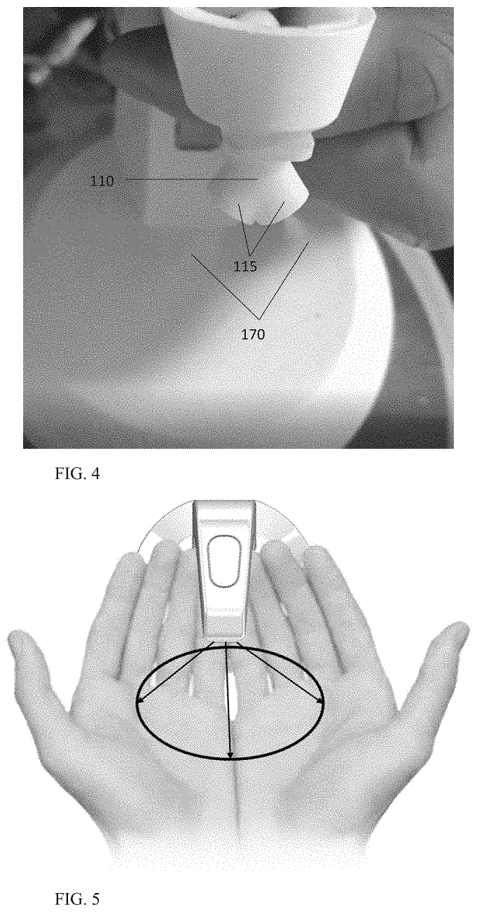

FIG. 3 illustrates a perspective view of hand sanitizer dispenser 100 including button 220 positioned on arm 130 configured for activating dispensing of sanitizing liquid, in accordance with one embodiment of the disclosure. Button 220 is configured to turn activate spray of sanitizing liquid 170. Button 220 can be positioned in different locations on arm 130 or around reservoir 180.

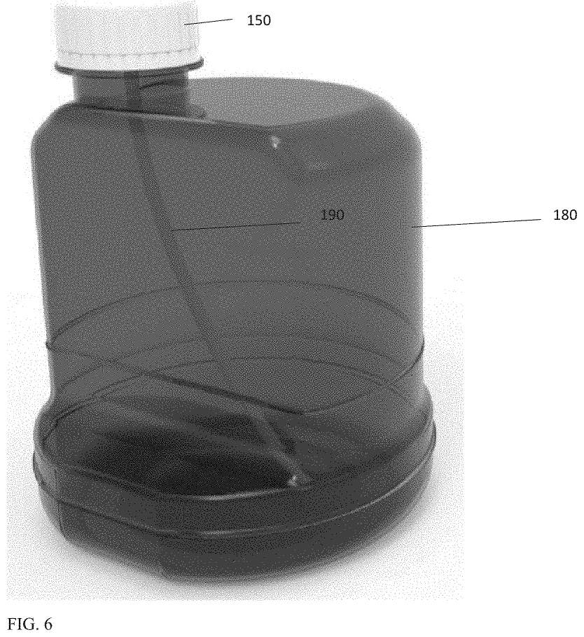

FIG. 4 illustrates an expanded perspective view of dual headed nozzle 110 of hand sanitizer dispenser 100, in accordance with an embodiment of the disclosure. In the illustrated embodiment, dual headed nozzle comprises two fan shaped protrusions 115 extending in opposite directions from a lower surface of the upper portion of the arm of hand sanitizer dispenser 100. In some embodiments, nozzle 105, 110 is positioned within a spray head. The spray head is configured to conceal the nozzle without limiting the width of spray achieved.

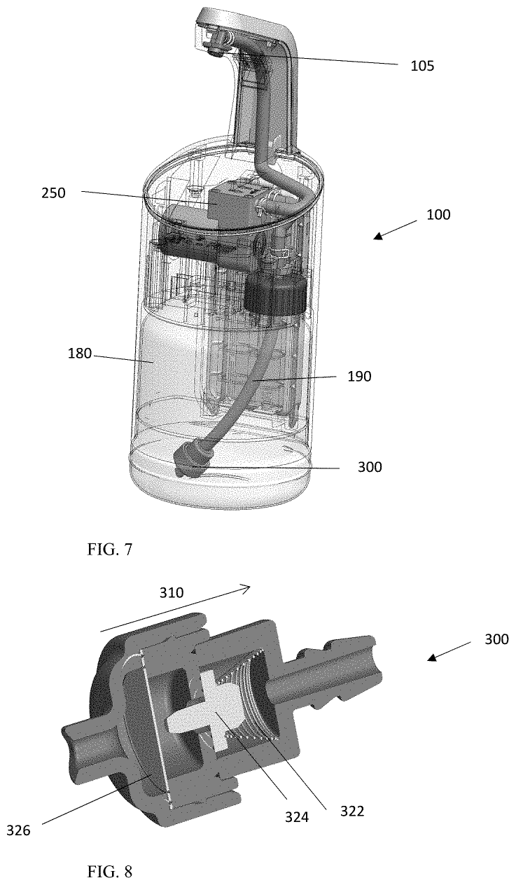

FIG. 5 illustrates a top view of a hand sanitizer dispenser, in accordance with one embodiment of the disclosure. FIG. 4 highlights an example range of coverage of sanitizing liquid dispensed onto a pair of a user's hands.

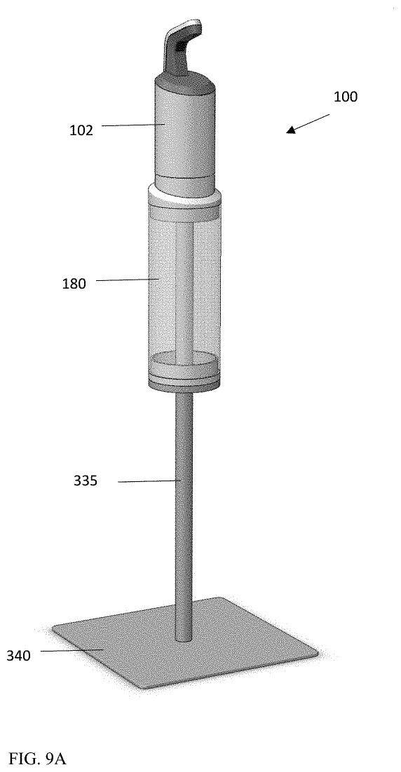

FIG. 6 illustrates reservoir 180 for containing sanitizing liquid 170, in accordance with an embodiment of the disclosure. Cap 150 is positioned on an upper portion of reservoir 180 and tubing 190 extending therefrom. In the illustrated embodiment, reservoir 180 is of irregular shape, with a protrusion at a lower end configured for fitting into a corresponding space of the housing 102 of hand sanitizer dispenser 100. In some embodiments, sanitizing liquid 170 is an alcohol-based sanitizing liquid. In some embodiments, sanitizing liquid 170 contains 75% ethyl alcohol. Reservoir 180 is removable from housing 102 and is refillable.

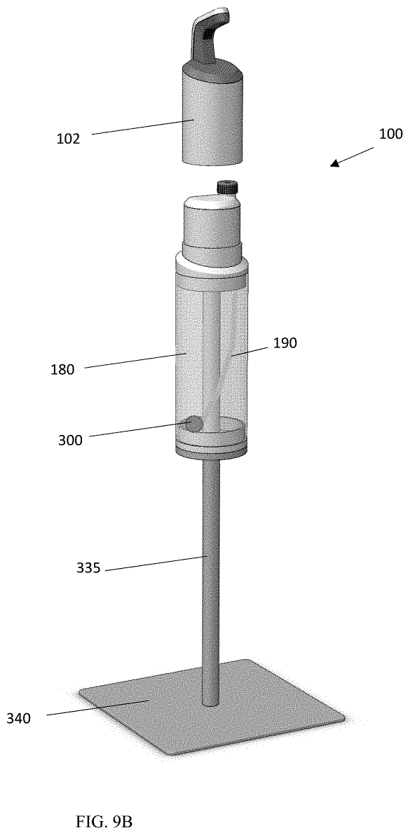

FIG. 7 illustrates a perspective interior view of a hand sanitizer dispenser 100, in accordance with one embodiment of the disclosure. In the illustrated embodiment, hand sanitizer dispenser 100 comprises pump 250 positioned centrally in the dispenser. In an embodiment, when an inlet for pump 250 is positioned on a lower surface of pump 250, liquid 170 can move down the internal passageways of hand sanitizer dispenser 100 due to gravity, resulting in air mixed with liquid 170 in the pipes. The result is that following a period of inactivity, for example for hours or days, an attempt to dispense liquid 170 from nozzle 105 may result in a reduced volume of dispensed liquid 170 due to the intermixed air.

In some embodiments, hand sanitizer dispenser 100 is configured to dispense a volume of liquid 170 between 0.4 mL and 1.7 mL, between 0.5 mL and 1.5 mL, or between 0.8 mL and 1.2 mL. In some embodiments, hand sanitizer dispenser 100 is configured to dispense liquid 170 over a period between 0.5 seconds and 2 seconds, between 0.6 seconds and 1.5 seconds, or between 0.7 seconds and 1 second.

One design challenge encountered in the development of this disclosure is the challenge of priming. If hand sanitizer dispenser 100 is not properly primed, then the full desired amount of sanitizer will not be properly dispensed, leading to potential consumer dissatisfaction.

One solution is the addition of one-way valve 300. In the embodiment illustrated in FIG. 7, one-way valve 300 is positioned at the end of tubing 190 and housed within reservoir 180. One-valve 300 acts to prevent liquid 170 from flowing downwards in the internal passageways of hand sanitizer dispenser 100 and ensures the internal passageways are full of liquid leading to a more complete dispensing of the desired volume of sanitizer during each use.

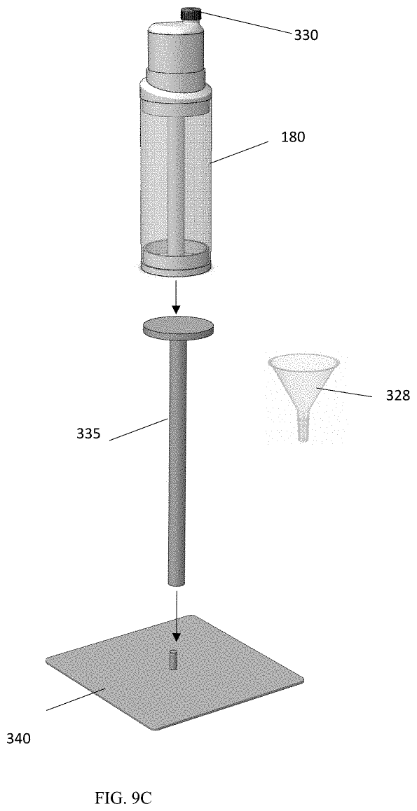

FIG. 8 illustrates a cross-sectional view of one-way valve 300, in accordance with one embodiment of the disclosure. In the illustrated embodiment, one-way valve 300 includes mesh screen 326 configured to prevent debris from entering the pump, seal 324, and spring 322. During use, liquid 170 travels along direction of flow 310. In some embodiments, mesh screen 326 comprises metal. In some embodiments, mesh screen 326 may have a mesh size of 140 (configured with 105 μm openings), 120 (configured with 125 μm openings), or 100 (configured with 150 μm openings). Mesh screens 326 are critical for diaphragm pumps that are susceptible to hair and particles.

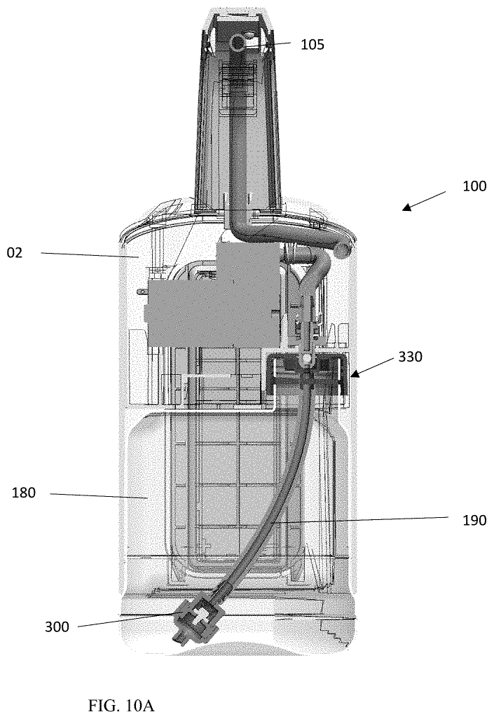

FIGS. 9A-C illustrate side views of hand sanitizer dispenser 100, in accordance with one embodiment of the disclosure. In the illustrated embodiment, hand sanitizer dispenser 100 is configured to be easily disassembled into different components. Hand sanitizer dispenser 100 is configured to be disassembled into housing 102, reservoir 180, support post 335, and stand 340. In some embodiments, as illustrated in FIG. 9C, reservoir 180, support post 335, and stand 340 are configured to be separated for packaging and/or shipment. In some embodiments, housing 102, reservoir 180, support post 335, and stand 340 are configured to be assembled with a screw connection. In other embodiments, a bayonet fitting or a rotational locking feature may be used for connecting housing 102, reservoir 180, support post 335, and/or stand 340. In some embodiments, reservoir 180 may connect to housing 102 with a quarter-turn, half-turn, or turn of a different angle. Alternative connection mechanisms include a snap fit, use of spring loaded catches, and a toe-in on one side with a corresponding catch on the other. In some embodiments, funnel 328 may be included for easy refilling of reservoir 180.

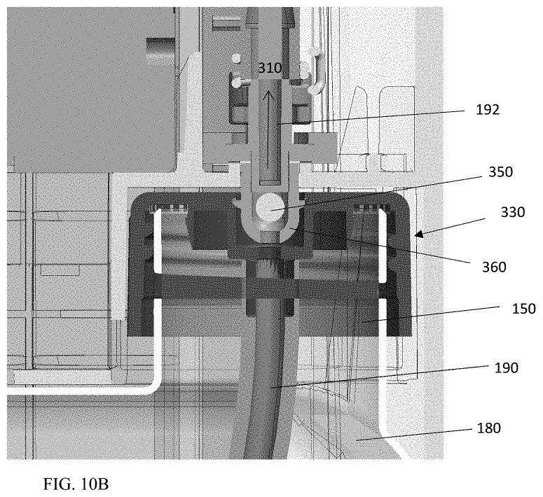

FIG. 10A illustrates a front interior view of hand sanitizer dispenser 100, in accordance with one embodiment of the disclosure. One-way valve 300 is illustrated at the end of tubing 190 and housed within reservoir 180. FIG. 10B illustrates a magnified view of connection point 330 between a reservoir and hand sanitizer dispenser housing 102 from FIG. 10A. During the process of dispensing liquid 170, flow occurs along direction of flow 310.

One design challenge encountered during the development of this disclosure is the prevention of backflow or dripping from portion 192 of the internal passageways. Following dispensing of liquid 170, some portion of liquid 170 may remain in the portion of tubing 192 above cap 150. Thus, when reservoir 180 is removed from housing 102, liquid 170 may flow out of tubing 192 undesirably. FIG. 10B illustrates an apparatus configured to prevent dripping when housing 102 is disconnected from reservoir 180. The illustrated embodiment provides ball valve 350 inside seal 360 that enters cap 150 at connection point 330. Thus, a seal is provided, the seal being configured to prevent and/or minimize backflow and/or dripping when reservoir 180 is removed from housing 102. During active dispensing of liquid 170, ball valve 350 is configured to move upwards in the direction of flow 310 without impact to the spray output. The foregoing description of example embodiments has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future-filed applications claiming priority to this application may claim the disclosed subject matter in a different manner and generally may include any set of one or more limitations as variously disclosed or otherwise demonstrated herein.

What is claimed is:

1. A system comprising:
a hand sanitizer dispenser, including
a reservoir configured to contain sanitizing liquid;
an arm extending above the reservoir;
a nozzle positioned on a lower surface of an upper portion of the arm, the nozzle configured for dispensing sanitizing liquid; and
a sensor for detection the presence of a user's hand,
wherein the arm is configured such that either one hand or two hands can enter a space between the reservoir and the arm.

2. The system of claim 1, wherein the nozzle is a dual headed nozzle.

3. The system of claim 2, wherein the dual headed nozzle comprises a pair of fan shaped protrusion configured to dispense sanitizing liquid.

4. The system of claim 1, further comprising a three-position button positioned on the arm.

5. The system of claim 4, wherein the three-position button is configured to switch between a set of dispensing modes, wherein each dispensing mode is targeted at one of liquid, gel, or foam.

6. The system of claim 1, wherein the nozzle is positioned inside a spray head.

7. The system of claim 3, further comprising a cap on an upper portion of the reservoir.

8. The system of claim 7, wherein a ball valve positioned in a seal is configured to facilitate a fluid-tight connection of the cap into a housing of the hand sanitizer dispenser.

9. The system of claim 8, wherein the ball valve is configured to minimize backflow of the sanitizing liquid.

10. The system of claim 7, further comprising tubing extending from the cap.

11. The system of claim 10, further comprising a one-way valve at the end of the tubing.

12. The system of claim 11, wherein the one-way valve comprises at least one of a mesh screen, a seal, and a spring.

13. The system of claim 8, further comprising a support post and a stand.

14. The system of claim 13, wherein at least one of the housing, the reservoir, the support post, and the stand are configured to be connected via a screw connection.

15. A method for dispensing sanitizing liquid, comprising:
providing a hand sanitizer dispenser according to claim 1;
sensing the presence of a user's hand; and
dispensing sanitizing liquid through the nozzle.

16. The method for dispensing sanitizing liquid of claim 15, wherein the nozzle is a dual-headed nozzle.

17. The method of claim 15, further comprising preventing a pump within the hand sanitizer dispenser from losing a strength of dispersal.

18. The method of claim 15, further comprising minimizing dripping during removal of a reservoir from the hand sanitizer dispenser.

19. A method for dispensing sanitizing liquid, comprising:
providing a hand sanitizer dispenser comprising a nozzle;
receiving user input from a button; and
dispensing sanitizing liquid through the nozzle upon receipt of user input.
